(19) United States
(12) Patent Application Publication
(10) Pub. No.: US 2025/0389651 A1
(43) Pub. Date: Dec. 25, 2025

(54) BIDIRECTIONAL REFLECTANCE SPECTROSCOPY MEASUREMENT DEVICE FOR TRACE MINERAL SAMPLES OF EXTRATERRESTRIAL OBJECTS

(71) Applicant: SHANGHAI INSTITUTE OF TECHNICAL PHYSICS CHINESE ACADEMY OF SCINCES, Hongkou (CN)

(72) Inventors: Zhiping He, Hongkou (CN); Jinning Li, Hongkou (CN); Rui Xu, Hongkou (CN); Aijun Jin, Hongkou (CN); Zhendong Wang, Hongkou (CN)

(21) Appl. No.: 19/235,752

(22) Filed: Jun. 12, 2025

(30) Foreign Application Priority Data
Jun. 24, 2024 (CN) 202410815880.1

(51) Int. Cl. G01N 21/47

(52) U.S. Cl. CPC G01N 21/4738; G01N 2021/4735; G01N 2201/0618; G01N 2201/0636

(57) ABSTRACT

A bidirectional reflectance spectroscopy measurement device for trace mineral samples of extraterrestrial objects includes a monochromatic illumination module, a microscopic spectroscopy measurement module, a sample carrying module and a control and data analysis module, wherein the monochromatic illumination module is configured with a light outlet, the microscopic spectroscopy measurement module is configured with a light inlet, the sample carrying module is located between the light outlet and the light inlet, and the control and data analysis module is electrically connected to the monochromatic illumination module, the microscopic spectroscopy measurement module and the sample carrying module respectively, the sample carrying module is configured to place and protect a sample to be measured, by synchronously controlling the monochromatic illumination module and the microscopic spectroscopy measurement module, spectral data of different spectral bands of a trace sample to be measured in the sample carrying module is obtained.

### CROSS-REFERENCE TO RELATED APPLICATIONS

This application claims the priority and benefit of Chinese patent application No. 202410815880.1, filed on Jun. 24, 2024. The entirety of Chinese patent application No. 202410815880.1 is hereby incorporated by reference herein and made a part of this specification.

### TECHNICAL FIELD

The disclosure relates to the technical field of spectral measurement, and in particular to a bidirectional reflectance spectroscopy measurement device for trace mineral samples of extraterrestrial objects.

### BACKGROUND ART

Extraterrestrial object sampling and returning is a new channel to obtain extraterrestrial mineral samples in addition to meteorites. The main minerals in the samples have different spectral characteristics in the visible to short-medium wave spectral range, and their reflection characteristics are bidirectional, that is, the reflection characteristics are related to the incident direction and detection direction of the light. Therefore, by measuring the bidirectional reflectance spectrum of the sample in this spectral range, the main mineral types and contents, the hydroxyl/water content, physical properties and space weathering characteristics in the sample can be distinguished and identified non-destructively, so as to analyze the main rock mineralogical characteristics and their evolutionary history in the sample, which is of great significance for studying the geological background information of the sample collection area and understanding the origin and evolution of extraterrestrial objects. In addition, it is also possible to simulate the illumination angle and detection angle of the on-orbit spectral instrument during detection, collect the spectral data of the measured sample in this state, provide a data processing method for the on-orbit detection data, and support the construction of an accurate material composition inversion model.

However, the number of samples returned from extraterrestrial objects is currently small and extremely precious. Therefore, the bidirectional reflectance spectroscopy measurement system needs less sample consumption, wider illumination and detection angle, and has a sample protection function. However, there is currently no bidirectional reflectance spectroscopy measurement device that can meet the testing needs of real trace samples of extraterrestrial objects (milligram level) and has the ability to protect the samples. It is impossible to provide scientific reference for the construction of the relationship between the spectral characteristics of materials of extraterrestrial objects and mineral chemistry, physics and space weathering information, as well as the remote sensing interpretation of the reflectance spectroscopy of extraterrestrial objects.

Therefore, there is a need to provide a bidirectional reflectance spectroscopy measurement device for trace mineral samples of extraterrestrial objects, which can realize Bidirectional reflectance spectroscopy measurement of trace samples of extraterrestrial objects and has sample protection capabilities compared with the existing technology.

### SUMMARY

The present disclosure solves the technical problems in the existing technology and provides a bidirectional reflectance spectroscopy measurement device for trace mineral samples of extraterrestrial objects.

In order to achieve the above-mentioned purpose of the disclosure,

the bidirectional reflectance spectroscopy measurement device for trace mineral samples of extraterrestrial objects includes a monochromatic illumination module, a microscopic spectroscopy measurement module, a sample carrying module and a control and data analysis module, wherein the monochromatic illumination module is configured with a light outlet, the microscopic spectroscopy measurement module is configured with a light inlet, the sample carrying module is located between the light outlet and the light inlet, and the control and data analysis module is electrically connected to the monochromatic illumination module, the microscopic spectroscopy measurement module and the sample carrying module respectively;

the sample carrying module is configured to place and protect a sample to be measured, the monochromatic illumination module is configured to output a total monochromatic light beam of millimeter scale and reflect it to a surface of the sample to be measured through the light outlet, the microscopic spectroscopy measurement module is configured to receive the light beam reflected from the surface of the sample to be measured and obtain spectral data of different spectral bands of the sample to be measured within a visible-mid-wave infrared spectral range.

Further, the monochromatic illumination module includes a first box, a halogen lamp, a spectral band classification assembly, a first reflection assembly and a reflection convergence mirror, wherein the light outlet is arranged on the first box, the halogen lamp, the spectral band classification assembly, the first reflection assembly and the reflection convergence mirror are all arranged inside the first box, the halogen lamp and the first reflection assembly are respectively arranged at two ends above the spectral band classification assembly, the reflection convergence mirror is located on a side of the first reflection assembly away from the halogen lamp, and the light outlet is located on a side of the reflection convergence mirror away from the first reflection assembly.

Furthermore, the first reflection assembly includes a first reflector and a second reflector, the first reflector is located above the second reflector, and the second reflector is located between the first reflector and the reflection convergence mirror; the reflection convergence mirror is configured to form the total monochromatic light beam of millimeter scale;

the first reflector is configured with a first reflection surface, which is arranged downward; the second reflector is configured with a second reflection surface, which is arranged upward.

Furthermore, the spectral band classification assembly includes a visible-infrared spectral band beam separation assembly and a short-wave-medium-wave infrared spectral band beam separation assembly, wherein the visible-infrared spectral band beam separation assembly is located above the short-wave-medium-wave infrared spectral band beam separation assembly, the visible-infrared spectral band beam separation assembly is configured to generate a first monochromatic light of a first set wavelength, and the short-wave-medium-wave infrared spectral band beam separation assembly is configured to generate a second monochromatic light of a second set wavelength, the visible-infrared spectral band beam separation assembly is also configured to transmit the second monochromatic light and reflect the first monochromatic light to the first reflection assembly, the short-wave-medium-wave infrared spectral band beam separation assembly is also configured to reflect the second monochromatic light to the first reflection assembly.

Furthermore, the visible-infrared spectral band beam separation assembly includes a first color separation plate, a first acousto-optic tunable filter and a third color separation plate, wherein the first color separation plate is located under the halogen lamp, the third color separation plate is located under the first reflector, and the first acousto-optic tunable filter is located between the first color separation plate and the third color separation plate;

the first color separation plate is configured to reflect the visible-infrared spectral band beam and transmit the short-wave-medium-wave infrared spectral band beam; the first acousto-optic tunable filter is configured to generate a first monochromatic light, and the third color separation plate is configured to reflect the first monochromatic light and transmit the second monochromatic light.

Furthermore, the short-wave-medium-wave infrared spectral band beam separation assembly includes a second color separation plate, a second acousto-optic tunable filter and a fourth color separation plate, wherein the second color separation plate is located under the first color separation plate, the fourth color separation plate is located below the third color separation plate, the second acousto-optic tunable filter is located between the second color separation plate and the fourth color separation plate;

the second color separation plate is configured to reflect the short-wave to medium-wave infrared spectral band beam, the second acousto-optic tunable filter is configured to generate a second monochromatic light, and the fourth color separation plate is configured to reflect the second monochromatic light.

Furthermore, an outer wall of the first box is also provided with a first drive assembly, the first drive assembly includes a first motor and a first bearing, the first motor and the first bearing are respectively arranged on the opposite outer walls of the first box, an output end of the first motor passes through the first box and is connected to the first bearing, the first drive assembly is configured to adjust an incident angle of a beam emitted to the surface of the sample to be measured, and an adjustment range of the incident angle of the beam emitted to the surface of the sample to be measured is −75°˜75°.

Further, the microscopic spectroscopy measurement module includes a second box, a reflection collimator, a second reflection assembly and a detection assembly based on spectral band, wherein the light inlet is arranged on the second box, the reflection collimator, the second reflection assembly and the detection assembly based on spectral band are all arranged inside the second box, the reflection collimator is located above the second reflection assembly, the light inlet and the detection assembly based on spectral band are respectively arranged at two ends below the second reflection assembly.

Furthermore, the second reflection assembly includes a third reflector and a fourth reflector, wherein the third reflector is located between the reflection collimator and the fourth reflector, the third reflector is configured with a third reflection surface, the third reflection surface is arranged upward, and the fourth reflector is configured with a fourth reflection surface, the fourth reflection surface is arranged downward.

Furthermore, the detection assembly based on spectral band includes a visible-near infrared beam detection assembly, a short-wave infrared beam detection assembly and a medium-wave infrared beam detection assembly arranged in sequence from top to bottom, the visible-near infrared beam detection assembly is configured to obtain the spectral data of sample to be measured in the visible-near infrared spectral band, the short-wave infrared beam detection assembly is configured to obtain the spectral data of the sample to be measured in the short-wave infrared spectral band, and the medium-wave infrared beam detection assembly is configured to obtain the spectral data of the sample to be measured in the medium-wave infrared spectral band.

Furthermore, the visible-near infrared beam detection assembly includes a fifth color separation plate, a first convergence mirror and a first detector, wherein the fifth color separation plate is located under the fourth reflector, the first convergence mirror is located between the fifth color separation plate and the first detector;

the fifth color separation plate is configured to reflect the visible-near infrared beam and transmit the beams in other spectral bands, and the first detector is configured to obtain the spectral data of the sample to be measured in the visible-near infrared spectral band.

Furthermore, the short-wave infrared beam detection assembly includes a sixth color separation plate, a second convergence mirror and a second detector, wherein the sixth color separation plate is located under the fifth color separation plate, and the second convergence mirror is located between the sixth color separation plate and the second detector;

the sixth color separation plate is configured to reflect the short-wave infrared beam and transmit the beams in other spectral bands, and the second detector is configured to obtain the spectral data of the sample to be measured in the short-wave infrared spectral band.

Furthermore, the medium-wave infrared beam detection assembly includes a seventh color separation plate, a third convergence mirror, and a third detector, wherein the seventh color separation plate is located under the sixth color separation plate, and the third convergence mirror is located between the seventh color separation plate and the third detector;

the seventh color separation plate is configured to reflect the medium-wave infrared beam, and the third detector is configured to obtain the spectral data of the sample to be measured in the medium-wave infrared spectral band.

Furthermore, an outer wall of the second box is also provided with a second drive assembly, the second drive assembly includes a second motor and a second bearing, the second motor and the second bearing are respectively arranged on the opposite outer walls of the second box, an output end of the second motor passes through the second box and is connected to the second bearing, the second drive assembly is configured to adjust a reflection angle of a beam reflected by the surface of the sample to be measured, and an adjustment range of the reflection angle is −75°˜75°.

Furthermore, a base plate is arranged at a lower part of the sample carrying module, the sample carrying module includes a platform, a plurality of sample vessels and a seal cover, wherein the platform is slidably connected to an upper wall of the base plate, the plurality of sample vessels are installed on an upper wall of the platform, and the upper wall of the platform is slidably connected to the seal cover.

Compared with the existing technology, the beneficial effects of the disclosure are that:
(1) the present disclosure synchronously controls the monochromatic illumination module and the microscopic spectroscopy measurement module to obtain spectral data of different spectral bands for a trace sample to be measured in the sample carrying module. The sample carrying module also has a protective function, thereby realizing a bidirectional reflectance spectroscopy measurement of trace samples of extraterrestrial objects with sample protection capability;
(2) the present disclosure controls the rotation of the monochromatic illumination module and the microscopic spectroscopy measurement module to adjust the range of the incident angle and the reflection angle, thereby measuring the spectral characteristics of the sample to be measured at different angles;
(3) the monochromatic illumination module of the present disclosure provides a wavelength-selectable monochromatic illumination source by controlling the high-speed switching of the sub-nanometer spectrum in the wavelength range of 0.4-3.2 μm. It adopts the spectral measurement scheme of “monochromatic modulated illumination+wide-band phase-locked weak signal extraction” and has higher background light suppression capability and system signal-to-noise ratio. In addition, the monochromatic illumination reduces the spectral irradiation of the sample surface, so that the sample surface temperature is more stable during the measurement process, and thereby realizing accurate separation of reflection and emission spectra.

### BRIEF DESCRIPTION OF THE DRAWINGS

FIG. 1 is a schematic diagram of the overall structure of the present disclosure.

FIG. 2 is a structural schematic diagram of the sample carrying module of the present disclosure.

### DETAILED DESCRIPTION

The technical scheme of the present disclosure will be clearly described below with reference to the accompanying drawings. Obviously, the described embodiments are not all embodiments of the present disclosure, and all other embodiments obtained by those of ordinary skill in the art without making any creative work shall fall within the scope of protection of the present disclosure. It should be noted that the orientations or positional relationships indicated by the terms “center”, “upper”, “lower”, “left”, “right”, “vertical” and “horizontal” etc. are based on the orientations or positional relationships shown in the accompanying drawings, and are only for the convenience of describing the present disclosure and simplifying the description, rather than indicating or implying that the device or element referred to must have a specific orientation, be constructed and operated in a specific orientation, and therefore cannot be understood as a limitation on the present disclosure.

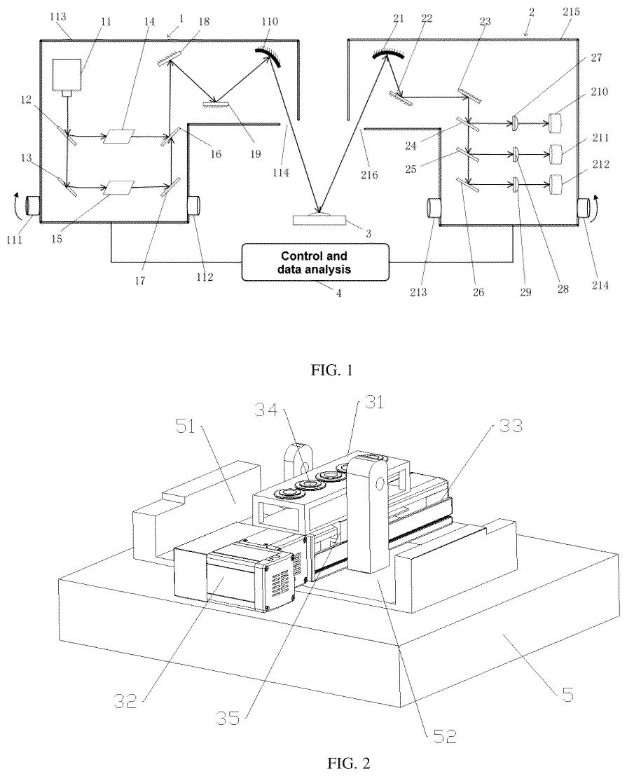

As shown in FIG. 1, the present disclosure provides a bidirectional reflectance spectroscopy measurement device for trace mineral samples of extraterrestrial objects, which includes a monochromatic illumination module 1, a microscopic spectroscopy measurement module 2, a sample carrying module 3 and a control and data analysis module 4. The sample carrying module 3 is located between the monochromatic illumination module 1 and the microscopic spectroscopy measurement module 2. The control and data analysis module 4 is electrically connected to the monochromatic illumination module 1, the microscopic spectroscopy measurement module 2 and the sample carrying module 3, respectively.

The monochromatic illumination module 1 is configured to generate monochromatic light in the visible-medium-wave infrared spectral band, converge the monochromatic beam to the millimeter scale, and output the monochromatic beam. The sample carrying module 3 is configured to place and protect the sample to be measured, and reflect the monochromatic beam output by the monochromatic illumination module 1. The microscopic spectroscopy measurement module 2 is configured to receive the monochromatic beam reflected by the sample carrying module 3, and obtain the spectral data of the sample in the visible-medium-wave infrared spectral band. The control and data analysis module 4 is configured to control the monochromatic illumination module 1 to generate the monochromatic beam, control the incident angle of the monochromatic beam, obtain the spectral data of the sample obtained by the microscopic spectroscopy measurement module 2, control the angle at which the sample reflects the monochromatic beam, and control the sample carrying module 3 to switch different samples.

The monochromatic illumination module 1 includes a first box 113, a halogen lamp 11, a spectral band classification assembly, a first reflection assembly, a reflection convergence mirror 110 and a first rotation assembly. The halogen lamp 11, the spectral band classification assembly, the first reflection assembly and the reflection convergence mirror 110 are all arranged inside the first box 113. The first rotation assembly is arranged on the outer side wall of the first box 113. The first box 113 is configured to be L-shaped. The first box 113 includes a first horizontal portion and a first vertical portion that are communicated to each other. A light outlet 114 is arranged at an end of the lower wall of the first horizontal portion away from the first vertical portion. The halogen lamp 11, the first reflection assembly and the reflection convergence mirror 110 are all located above the spectral band classification assembly and the light outlet 114. The halogen lamp 11 and the first reflection assembly are located at two ends above the spectral band classification assembly. The halogen lamp 11 is arranged away from the light outlet 114 relative to the first reflection assembly. The reflection convergence mirror 110 is located on the side of the first reflection assembly away from the spectral band classification assembly. The reflection convergence mirror 110 is located between the first reflection assembly and the light outlet 114. The halogen lamp 11 is located above the spectral band classification assembly, and is configured to generate the visible-medium-wave infrared broadband light.

The first reflection assembly includes a first reflector 18 and a second reflector 19. The first reflector 18 is located above the second reflector 19, the second reflector 19 is located below the reflection convergence mirror 110, and the second reflector 19 is located between the first reflector 18 and the reflection convergence mirror 110. The first reflector 18 is configured with a first reflection surface, the first reflection surface is arranged downward, and the first reflection surface is arranged obliquely downward from an end close to the second reflector 19 to an end away from the second reflector 19. The second reflector 19 is configured with a second reflection surface, the second reflection surface is arranged facing directly upward. The reflection convergence mirror 110 is configured with a convergence surface. The convergence surface is arc-shaped, arranged downward, concave, and arranged obliquely downward from an end close to the second reflector 19 to an end away from the second reflector 19.

The spectral band classification assembly includes a visible-near infrared spectral band beam separation assembly and a short-wave-medium-wave infrared spectral band beam separation assembly. The visible-near infrared spectral band beam separation assembly is located above the short-wave-medium-wave infrared spectral band beam separation assembly. The visible-near infrared spectral band beam separation assembly includes a first color separation plate 12, a first acousto-optic tunable filter 14 and a third color separation plate 16. The first color separation plate 12 is located under the halogen lamp 11, the third color separation plate 16 is located under the first reflector 18, the first acousto-optic tunable filter 14 is located between the first color separation plate 12 and the third color separation plate 16, the first color separation plate 12 is inclined upward from an end close to the first acousto-optic tunable filter 14 to an end away from the first acousto-optic tunable filter 14, and the third color separation plate 16 is inclined upward from an end close to the first acousto-optic tunable filter 14 to an end away from the first acousto-optic tunable filter 14. The short-wave-medium-wave infrared spectral beam separation assembly includes a second color separation plate 13, a second acousto-optic tunable filter 15 and a fourth color separation plate 17, the second color separation plate 13 is located under the first color separation plate 12, the fourth color separation plate 17 is located under the third color separation plate 16, the second acousto-optic tunable filter 15 is located between the second color separation plate 13 and the fourth color separation plate 17, the second color separation plate 13 is arranged obliquely upward from an end close to the second acousto-optic tunable filter 15 to an end away from the second acousto-optic tunable filter 15, and the fourth color separation plate 17 is arranged obliquely upward from an end close to the second acousto-optic tunable filter 15 to an end away from the second acousto-optic tunable filter 15.

The visible-medium-wave infrared broadband light generated by the halogen lamp 11 is directed toward the first color separation plate 12, and the first color separation plate 12 reflects the visible-near infrared spectral band beam to the first acousto-optic tunable filter 14. At the same time, the first color separation plate 12 transmits the short-wave-medium-wave infrared spectral band beam, and the short-wave-medium-wave infrared spectral band beam is directed toward the second color separation plate 13, and reflected by the second color separation plate 13 to the second acousto-optic tunable filter 15. The first acousto-optic tunable filter 14 splits the visible-near infrared spectral band beam to form a first monochromatic light with a first set wavelength, and directs the first monochromatic light to the third color separation plate 16. The first monochromatic light is reflected by the third color separation plate 16 onto the first reflection surface of the first reflector 18, and at the same time, the second acousto-optic tunable filter 15 splits the short-wave-medium-wave infrared spectral band beam to form a second monochromatic light with a second set wavelength, and directs the second monochromatic light to the fourth color separation plate 17. The second monochromatic light is reflected by the fourth color separation plate 17 and passes through the third color separation plate 16, and then reflected onto the first reflection surface of the first reflector 18. The first monochromatic light and the second monochromatic light reflected by the first reflector 18 and the second reflector 19 in sequence are directed to the convergence surface of the reflection convergence mirror 110, and then converged into a total monochromatic light of millimeter scale. The total monochromatic light is reflected by the reflection convergence mirror 110, then reflected through the light outlet 114 to the surface of the sample to be measured of the sample carrying module 3.

The first drive assembly includes a first motor 111 and a first bearing 112, which are respectively arranged on the opposite outer walls of the first vertical portion of the first box 113. The output of the first motor 111 passes through the first box 113 and is connected to the first bearing 112. The output of the first motor 111 is fixedly connected to the first box 113. The first drive assembly drives the first box 113 to rotate in a direction perpendicular to FIG. 1, so that the incident angle adjustment range of the total monochromatic light is −75°˜75°.

The microscopic spectroscopy measurement module 2 includes a second box 215, a reflection collimator 21, a second reflection assembly, a detection assembly based on spectral band and a second drive assembly. The reflection collimator 21, the second reflection assembly and the detection assembly based on spectrum band are all arranged inside the second box 215, and the second drive assembly is arranged on the outer wall of the second box 215. The second box 215 is configured to be L-shaped, and includes a second horizontal portion and a second vertical portion communicated with each other. A light inlet 216 is provided at an end of the lower wall of the second horizontal portion away from the second vertical portion. The first horizontal portion and the second horizontal portion are arranged opposite to each other. The reflection collimator 21 is arranged above the second reflection assembly. The second reflection assembly is located above the detection assembly based on spectral band.

The second reflection assembly includes a third reflector 22 and a fourth reflector 23. The third reflector 22 is located below the reflection collimator 21, and the fourth reflector 23 is located to the right of the third reflector 22. The third reflector 22 is configured with a third reflection surface, which is arranged upward, and the third reflection surface is inclined upward from an end close to the fourth reflector 23 to an end away from the fourth reflector 23. The fourth reflector 23 is configured with a fourth reflection surface, which is arranged downward, and the fourth reflection surface is inclined downward from an end close to the third reflector 22 to an end away from the third reflector 22. The reflection collimator 21 is configured with a collimation surface, which is arranged downward, arc-shaped, and concave.

The detection assembly based on spectral band includes a visible-near infrared beam detection assembly, a short-wave infrared beam detection assembly and a medium-wave infrared beam detection assembly. The short-wave infrared beam detection assembly is located below the visible-near infrared beam detection assembly, and the medium-wave infrared beam detection assembly is located below the short-wave infrared beam detection assembly. The visible-near infrared beam detection assembly includes a fifth color separation plate 24, a first convergence mirror 27 and a first detector 210 arranged in sequence from left to right, the short-wave infrared beam detection assembly includes a sixth color separation plate 25, a second convergence mirror 28 and a second detector 211 arranged in sequence from left to right, and the medium-wave infrared beam detection assembly includes a seventh color separation plate 26, a third convergence mirror 29 and a third detector 212 arranged in sequence from left to right. The sixth color separation plate 25 is located under the fifth color separation plate 24, the seventh color separation plate 26 is located under the sixth color separation plate 25, the second convergence mirror 28 is located under the first convergence mirror 27, the third convergence mirror 29 is located under the second convergence mirror 28, the second detector 211 is located under the first detector 210, and the third detector 212 is located under the second detector 211. The fifth color separation plate 24 is inclined upward from an end close to the first convergence mirror 27 to an end away from the first convergence mirror 27, the sixth color separation plate 25 is color separation film upward from an end close to the second convergence mirror 28 to an end away from the second convergence mirror 28, and the seventh color separation plate 26 is inclined upward from an end close to the third convergence mirror 29 to an end away from the third convergence mirror 29.

The beam reflected by the surface of the sample to be measured of the sample carrying module 3 enters the second box 215 through the light inlet 216 and then is directed to the collimation surface of the reflection collimator 21 for collimation. The collimated beam is reflected by the third reflector 22 and the fourth reflector 23 in sequence. The fifth color separation plate 24 reflects the visible-near infrared beam in the beam directed thereto to the first convergence mirror 27 and then directed to the first detector 210 for detection, thereby obtaining the spectral data of the sample in the visible-near infrared spectral band. The fifth color separation plate 24 transmits the beams of other spectral bands directed thereto, the sixth color separation plate 25 reflects the short-wave infrared beam in the beam directed thereto to the second convergence mirror 28 and then directs it to the inside of the second detector 211 for detection, thereby obtaining the spectral data of the sample in the short-wave infrared spectral band. The sixth color separation plate 25 transmits the beams of other spectral bands directed thereto, and the seventh color separation plate 26 reflects the medium-wave infrared beam directed thereto to the third convergence mirror 29 and then directs it to the inside of the third detector 212 for detection, thereby obtaining the spectral data of the sample in the medium-wave infrared spectral band.

The second drive assembly includes a second motor 213 and a second bearing 214, which are respectively arranged on the opposite outer walls of the second vertical portion. The second drive assembly drives the second box 215 to rotate in a direction perpendicular to FIG. 1. The output of the second motor 213 passes through the second box 215 and is connected to the second bearing 214. The output of the second motor 213 is fixedly connected to the second box 215, so that the adjustment range of the reflection angle of the beam reflected by the sample is −75°˜75°. The angle between the incident beam on the sample and the reflected beam passing through the sample is the phase angle. Therefore, the effective range of the phase angle is 10°˜150°.

As shown in FIG. 2, the bidirectional reflectance spectroscopy measurement device for trace mineral samples of extraterrestrial objects provided by the present disclosure also includes a base plate 5, on which a first placement cavity 51 and a second placement cavity 52 are provided. The monochromatic illumination module 1 is placed in the first placement cavity 51, and the microscopic spectroscopy measurement module 2 is placed in the second placement cavity 52. The first motor 111, the second motor 213, the first bearing 112, and the second bearing 214 are all fixedly connected to the base plate 5. The sample carrying module 3 is arranged between the first placement cavity 51 and the second placement cavity 52, and the sample carrying module 3 is arranged on the upper wall of the base plate 5. The sample carrying module 3 includes a platform 31, a seal cover, and a plurality of sample vessels 34. The platform 31 is slidably connected to the upper wall of the base plate 5. The plurality of sample vessels 34 are installed on the upper wall of the platform 31 along its length direction. The upper wall of the platform 31 is slidably connected to the seal cover, which can cover the plurality of sample vessels 34. The structure of the seal cover is not shown in FIG. 2. The upper wall of the base plate 5 is fixedly connected to a slide rail 33, and the slide rail 33 is slidably connected to the slider 35. The platform 31 is fixedly connected to the upper wall of the slider 35. A drive motor 32 is also fixedly connected on the base plate. The axis of the output of the drive motor 32 can be parallel or perpendicular to the length direction of the slide rail 33. When the axis of the output of the drive motor 32 is perpendicular to the slide rail 33, the output of the drive motor 32 is connected with a steering gear. The output of the drive motor 32 or the output of the steering gear connected to the output of the drive motor 32 is connected to one end of a screw, and the other end of the screw is rotatably connected to the opposite side wall of the slide rail 33 where the drive motor 32 is provided. The screw passes through the slider 35, and the screw is threadedly connected to the slider 35.

The sample carrying module 3 is equipped with a variety of standard samples for calibration, including standard wavelength materials (dysprosium oxide, erbium oxide, etc.), reflectance standard plate kit, polytetrafluoroethylene and gold-plated standard diffuse reflectance calibration samples. The seal cover is provided to meet the protection needs of the lunar soil such as moisture and oxidation protection.

The control and data analysis module 4 includes a control module and a data analysis module. The control module is respectively connected to the first drive assembly, the second drive assembly, the first acousto-optic tunable filter 14, the second acousto-optic tunable filter 15, the first detector 210, the second detector 211, and the third detector 212. The control module is configured to adjust the rotation angle of the first drive assembly, thereby adjusting the incident angle of the beam on the sample surface. The control module is also configured to adjust the rotation angle of the second drive assembly, thereby adjusting the reflection angle of the beam reflected from the sample surface. The control module is also configured to adjust the wavelength output by the first acousto-optic tunable filter 14 and the second acousto-optic tunable filter 15. The control module is also configured to control the spectral bands of the beams detected by the first detector 210, the second detector 211, and the third detector 212 respectively. The spectral data of the sample in different spectral bands are stored in the data analysis module, and the data analysis module performs photometric model calculation on the spectral data through spectral processing tools such as spectral smoothing, resampling, envelope removal, MGM fitting, and supports the inversion of identification and content of lunar soil minerals, the inversion of lunar soil physical properties, and the research on lunar remote sensing calibration methods according to bidirectional reflectance data of lunar soil samples based on spectral analysis capabilities such as material composition analysis of MGM.

The present disclosure synchronously controls the monochromatic illumination module 1 and the microscopic spectroscopy measurement module 2 to realize the imaging recording of the sample observation area and obtain the spectral characteristics of the sample to be measured under specific illumination conditions. In addition, the rotation of the monochromatic illumination module 1 and the microscopic spectroscopy measurement module 2 are controlled to adjust the range of the incident angle and the reflection angle, thereby measuring the spectral characteristics of the sample to be measured at different angles. The monochromatic illumination module 1 provides a wavelength-selectable monochromatic illumination source by controlling the high-speed switching of the sub-nanometer spectrum in the wavelength range of 0.4-3.2 μm. It adopts the spectral measurement scheme of “monochromatic modulated illumination+wide-band phase-locked weak signal extraction” and has higher background light suppression capability and system signal-to-noise ratio. In addition, the monochromatic illumination reduces the spectral irradiation of the sample surface, so that the sample surface temperature is more stable during the measurement process, and thereby realizing accurate separation of reflection and emission spectra. A bidirectional reflectance spectroscopy measurement of trace samples of extraterrestrial objects with sample protection capability is realized.

It should be noted that the above content is only used to illustrate the present disclosure, rather than to limit the scope of protection of the present disclosure. Simple modifications or equivalent substitutions of the present disclosure by ordinary skilled in the art do not deviate from the essence and scope of the present disclosure.

What is claimed is:

1. A bidirectional reflectance spectroscopy measurement device for trace mineral samples of extraterrestrial objects, comprising a monochromatic illumination module, a microscopic spectroscopy measurement module, a sample carrying module and a control and data analysis module, wherein the monochromatic illumination module is configured with a light outlet, the microscopic spectroscopy measurement module is configured with a light inlet, the sample carrying module is located between the light outlet and the light inlet, and the control and data analysis module is electrically connected to the monochromatic illumination module, the microscopic spectroscopy measurement module and the sample carrying module respectively;
the sample carrying module is configured to place and protect a sample to be measured, the monochromatic illumination module is configured to output a total monochromatic light beam of millimeter scale and reflect the total monochromatic light beam of millimeter scale to a surface of the sample to be measured through the light outlet, and the microscopic spectroscopy measurement module is configured to receive the light beam reflected from the surface of the sample to be measured and obtain spectral data of different spectral bands of the sample to be measured within a visible-mid-wave infrared spectral range.

2. The bidirectional reflectance spectroscopy measurement device for trace mineral samples of extraterrestrial objects according to claim 1, wherein the monochromatic illumination module comprises a first box, a halogen lamp, a spectral band classification assembly, a first reflection assembly and a reflection convergence mirror, the light outlet is arranged on the first box, the halogen lamp, the spectral band classification assembly, the first reflection assembly and the reflection convergence mirror are all arranged inside the first box, the halogen lamp and the first reflection assembly are respectively arranged at two ends above the spectral band classification assembly, the reflection convergence mirror is located on a side of the first reflection assembly away from the halogen lamp, and the light outlet is located on a side of the reflection convergence mirror away from the first reflection assembly.

3. The bidirectional reflectance spectroscopy measurement device for trace mineral samples of extraterrestrial objects according to claim 2, wherein the first reflection assembly comprises a first reflector and a second reflector, the first reflector is located above the second reflector, the second reflector is located between the first reflector and the reflection convergence mirror, and the reflection convergence mirror is configured to form the total monochromatic light beam of millimeter scale;
the first reflector is configured with a first reflection surface, which is arranged downward, and the second reflector is configured with a second reflection surface, which is arranged upward.

4. The bidirectional reflectance spectroscopy measurement device for trace mineral samples of extraterrestrial objects according to claim 3, wherein the spectral band classification assembly comprises a visible-infrared spectral band beam separation assembly and a short-wave-medium-wave infrared spectral band beam separation assembly, the visible-infrared spectral band beam separation assembly is located above the short-wave-medium-wave infrared spectral band beam separation assembly, the visible-infrared spectral band beam separation assembly is configured to generate a first monochromatic light of a first set wavelength, the short-wave-medium-wave infrared spectral band beam separation assembly is configured to generate a second monochromatic light of a second set wavelength, the visible-infrared spectral band beam separation assembly is also configured to transmit the second monochromatic light and reflect the first monochromatic light to the first reflection assembly, and the short-wave-medium-wave infrared spectral band beam separation assembly is also configured to reflect the second monochromatic light to the first reflection assembly.

5. The bidirectional reflectance spectroscopy measurement device for trace mineral samples of extraterrestrial objects according to claim 4, wherein the visible-infrared spectral band beam separation assembly comprises a first color separation plate, a first acousto-optic tunable filter and a third color separation plate, the first color separation plate is located under the halogen lamp, the third color separation plate is located under the first reflector, and the first acousto-optic tunable filter is located between the first color separation plate and the third color separation plate;
the first color separation plate is configured to reflect a visible-infrared spectral band beam and transmit a short-wave-medium-wave infrared spectral band beam, the first acousto-optic tunable filter is configured to generate the first monochromatic light, and the third color separation plate is configured to reflect the first monochromatic light and transmit the second monochromatic light.

6. The bidirectional reflectance spectroscopy measurement device for trace mineral samples of extraterrestrial objects according to claim 5, wherein the short-wave-medium-wave infrared spectral band beam separation assembly comprises a second color separation plate, a second acousto-optic tunable filter and a fourth color separation plate, the second color separation plate is located under the first color separation plate, the fourth color separation plate is located below the third color separation plate, and the second acousto-optic tunable filter is located between the second color separation plate and the fourth color separation plate;
the second color separation plate is configured to reflect the short-wave-medium-wave infrared spectral band beam, the second acousto-optic tunable filter is configured to generate the second monochromatic light, and the fourth color separation plate is configured to reflect the second monochromatic light.

7. The bidirectional reflectance spectroscopy measurement device for trace mineral samples of extraterrestrial objects according to claim 2, wherein an outer wall of the first box is also provided with a first drive assembly, the first drive assembly comprises a first motor and a first bearing, the first motor and the first bearing are respectively arranged on opposite outer walls of the first box, an output end of the first motor passes through the first box and is connected to the first bearing, the first drive assembly is configured to adjust an incident angle of the total monochromatic light beam emitted to the surface of the sample to be measured, and an adjustment range of the incident angle of the total monochromatic light beam emitted to the surface of the sample to be measured is −75°-75°.

8. The bidirectional reflectance spectroscopy measurement device for trace mineral samples of extraterrestrial objects according to claim 1, wherein the microscopic spectroscopy measurement module comprises a second box, a reflection collimator, a second reflection assembly and a detection assembly based on spectral band, the light inlet is arranged on the second box, the reflection collimator, the second reflection assembly and the detection assembly based on spectral band are all arranged inside the second box, the reflection collimator is located above the second reflection assembly, and the light inlet and the detection assembly based on spectral band are respectively arranged at two ends below the second reflection assembly.

9. The bidirectional reflectance spectroscopy measurement device for trace mineral samples of extraterrestrial objects according to claim 8, wherein the second reflection assembly comprises a third reflector and a fourth reflector, the third reflector is located between the reflection collimator and the fourth reflector, the third reflector is configured with a third reflection surface, the third reflection surface is arranged upward, the fourth reflector is configured with a fourth reflection surface, and the fourth reflection surface is arranged downward.

10. The bidirectional reflectance spectroscopy measurement device for trace mineral samples of extraterrestrial objects according to claim 9, wherein the detection assembly based on spectral band comprises a visible-near infrared beam detection assembly, a short-wave infrared beam detection assembly and a medium-wave infrared beam detection assembly arranged in sequence from top to bottom, the visible-near infrared beam detection assembly is configured to obtain the spectral data of the sample to be measured in a visible-near infrared spectral band, the short-wave infrared beam detection assembly is configured to obtain the spectral data of the sample to be measured in a short-wave infrared spectral band, and the medium-wave infrared beam detection assembly is configured to obtain the spectral data of the sample to be measured in a medium-wave infrared spectral band.

11. The bidirectional reflectance spectroscopy measurement device for trace mineral samples of extraterrestrial objects according to claim 10, wherein the visible-near infrared beam detection assembly comprises a fifth color separation plate, a first convergence mirror and a first detector, the fifth color separation plate is located under the fourth reflector, and the first convergence mirror is located between the fifth color separation plate and the first detector;
the fifth color separation plate is configured to reflect a visible-near infrared beam and transmit beams in other spectral bands, and the first detector is configured to obtain the spectral data of the sample to be measured in the visible-near infrared spectral band.

12. The bidirectional reflectance spectroscopy measurement device for trace mineral samples of extraterrestrial objects according to claim 11, wherein the short-wave infrared beam detection assembly comprises a sixth color separation plate, a second convergence mirror and a second detector, the sixth color separation plate is located under the fifth color separation plate, and the second convergence mirror is located between the sixth color separation plate and the second detector;
the sixth color separation plate is configured to reflect a short-wave infrared beam and transmit the beams in other spectral bands, and the second detector is configured to obtain the spectral data of the sample to be measured in the short-wave infrared spectral band.

13. The bidirectional reflectance spectroscopy measurement device for trace mineral samples of extraterrestrial objects according to claim 12, wherein the medium-wave infrared beam detection assembly comprises a seventh color separation plate, a third convergence mirror, and a third detector, the seventh color separation plate is located under the sixth color separation plate, and the third convergence mirror is located between the seventh color separation plate and the third detector;
the seventh color separation plate is configured to reflect a medium-wave infrared beam, and the third detector is configured to obtain the spectral data of the sample to be measured in the medium-wave infrared spectral band.

14. The bidirectional reflectance spectroscopy measurement device for trace mineral samples of extraterrestrial objects according to claim 8, wherein an outer wall of the second box is also provided with a second drive assembly, the second drive assembly comprises a second motor and a second bearing, the second motor and the second bearing are respectively arranged on opposite outer walls of the second box, an output end of the second motor passes through the second box and is connected to the second bearing, the second drive assembly is configured to adjust a reflection angle of the total monochromatic light beam reflected by the surface of the sample to be measured, and an adjustment range of the reflection angle is −75°-75°.

15. The bidirectional reflectance spectroscopy measurement device for trace mineral samples of extraterrestrial objects according to claim 1, wherein a base plate is arranged at a lower part of the sample carrying module, the sample carrying module comprises a platform, a plurality of sample vessels and a seal cover, wherein the platform is slidably connected to an upper wall of the base plate, the plurality of sample vessels are installed on an upper wall of the platform, and the upper wall of the platform is slidably connected to the seal cover.
